(19) United States
(12) Patent Application Publication
(10) Pub. No.: US 2006/0180159 A1
(43) Pub. Date: Aug. 17, 2006

(54) Cracked heel protector

(76) Inventor: Jennifer Duvoe, Roseville, CA (US)

Correspondence Address: Mark C. Jacobs, 3033 El Camino Avenue, Sacramento, CA, 95821, US

(21) Appl. No.: 11/055,800

(22) Filed: Feb. 11, 2005

(51) Int. Cl. A61F 13/00; A61F 13/06

(52) U.S. Cl. 128/892; 128/889

(57) ABSTRACT

A fabric device for wear over a user's heel to protect a cracked or inflamed heel, whether being medically treated or not, to protect the heel from further injury and discomfort. The device inhibits the undesired spread of applied medicaments to clothing such as socks, or shoe interiors, and to bedding during sleep. A version is formed by cutting and folding a T-shaped unit by bending the base of the Tee upward, to the top of the Tee's extremities into a U-shaped configuration, and folding and joining the two parts of the Tee together. Or using a rectangle, the side midpoints are folded to spaced on top locations, stitched downwardly to yield a second version body portion having an open top and front and a stitched rear. The edges may be bound as with surging, and a closure strap, attached to the body at about a 45-degree angle.

### FIELD OF THE INVENTION

This invention pertains to a fabric, cloth, or non-woven fabric heel cover to be worn by persons who have cracked heels as well as by persons who are treating the cracked heel of their foot with Vaseline® or other brands of petroleum jelly or skin balms intended to soften the skin. The device of this invention is worn by this second group of people to inhibit and hopefully prevent staining of sheets and rugs by the treatment medicament.

### BACKGROUND OF THE INVENTION

Some men and many women suffer from dry foot heels. These dry heels both at the vertical surface and the horizontal surface can be tender and sore. Men are subject to this problem as well but since women tend to wear more open heeled shoes the problem is more acute for them, since socks can damper the discomfort and hide the appearance of cracked heels.

One way to treat dry and cracked heels is to rub them with Vaseline® or other liquid balms, which provide temporary relief and improve the appearance of the cracked heel. This device is worn to cover the heel to prevent the transference of the medicament to other surfaces. The material employed for the device was specifically chosen to be substantially nonabsorbent thereby maximum benefit of the medicament can be received.

Some people may choose not to medicate their heel for one reason or another in such instance the device of this invention provides comfort in walking about the house, particularly to those who walk barefoot on rugs and floors.

The invention accordingly comprises the device possessing the features, properties, the selection of components which are amplified in the following detailed disclosure, and the scope of the application of which will be indicated in the appended claims.

For a fuller understanding of the nature and objects of the invention, reference should be made to the following detailed description, taken in conjunction with the accompanying drawings.

### BRIEF SUMMARY OF THE INVENTION

A heel cover to be fastened over the instep of the wearer provides protection to the cracked heels of wearers who tend to walk barefoot around the house, in the yard, and on the beach. When the heel is treated with Vaseline® or other medicament to soften the dry skin that causes cracked heels, this device prevents transference of the medicament to bedding, rugs, and other surfaces. Maximum benefit of the medicament is achievable especially since the cloth preferred is nonabsorptive of the medicament dwelling on the wearer's heel.

It is a first object to provide a heel protector to be worn by persons having cracked or inflamed heels.

It is a second object to provide a heel protector that can be worn over medicaments without fear of “bleed through.”

It is a third object to provide a heel protector that can be worn with socks.

It is a fourth object to provide a heel protector that is easy to put on using but a single strap to secure it in place.

It is a fifth object to provide a heel protector made of two layers of quilted fabric having a batting layer interposed.

It is a sixth object to provide 2 embodiments of this invention that are substantially similar, the differences being in the assembly thereof.

These and other objects of the invention will in part be obvious and will in part appear hereinafter. The invention accordingly comprises the details of the disclosure as set forth herein with particular reference to the appended drawings.

### BRIEF DESCRIPTION OF FIGURES

FIG. 1 is a layout of the fabric member used to construct the device of this invention's first embodiment.

FIG. 2 is a left side elevational view of a closed right closing device of this invention's first embodiment.

FIG. 3 is a top front perspective view of a first version left closing device according to this invention.

FIG. 4 is a left side elevational view of a first version left closing device of this invention.

FIG. 5 is a view similar to FIG. 2 but in an open non-closed position.

FIG. 6 is a rear elevational view of a device of this invention's first version.

FIG. 7 is a right side elevational view of the device of FIG. 2 in open position.

FIG. 8 is a left front perspective view showing a first embodiment device of this invention in a disposed position on the heel of the wearer.

FIG. 9 is a top front perspective of the same wearer's instep area.

FIG. 10 is a graphical depiction of the assembly of the device of FIG. 2 in open position.

FIG. 11 is a diagrammatic view intended to show how the surging binds the fabric for both embodiments.

FIG. 12 is a view related to FIG. 6, but which view arises when the device is in use.

FIG. 13 is a plan view of another suitable closure means of securing the strap of this invention.

FIG. 14 is a side elevational view that illustrates a variant hook and loop closure for securing the device to the heel of the wearer.

FIG. 15 is a plan view of the layout of the fabric member used to construct the second embodiment of the device of this invention.

FIG. 16 is a diagrammatic representation of the folding procedure to assemble the fabric member of FIG. 15 into the second embodiment.

FIG. 17 is a left side elevational view of a right closing second embodiment of the invention.

FIG. 18 is a right side view of the device of FIG. 17.

FIG. 19 is a view similar to FIG. 17, but with strap in open position.

FIG. 20 is a view similar to FIG. 8, but showing the second embodiment being worn by the user.

### DESCRIPTION OF THE PREFERRED EMBODIMENT

The device of this invention is discussed with reference to two embodiments or versions. The two versions are used in like manner. The differences lie only in the mode of folding and stitching of the fabric member from which the device is created. The benefits and for that matter the detriments of one over the other are trivial but will be discussed infra.

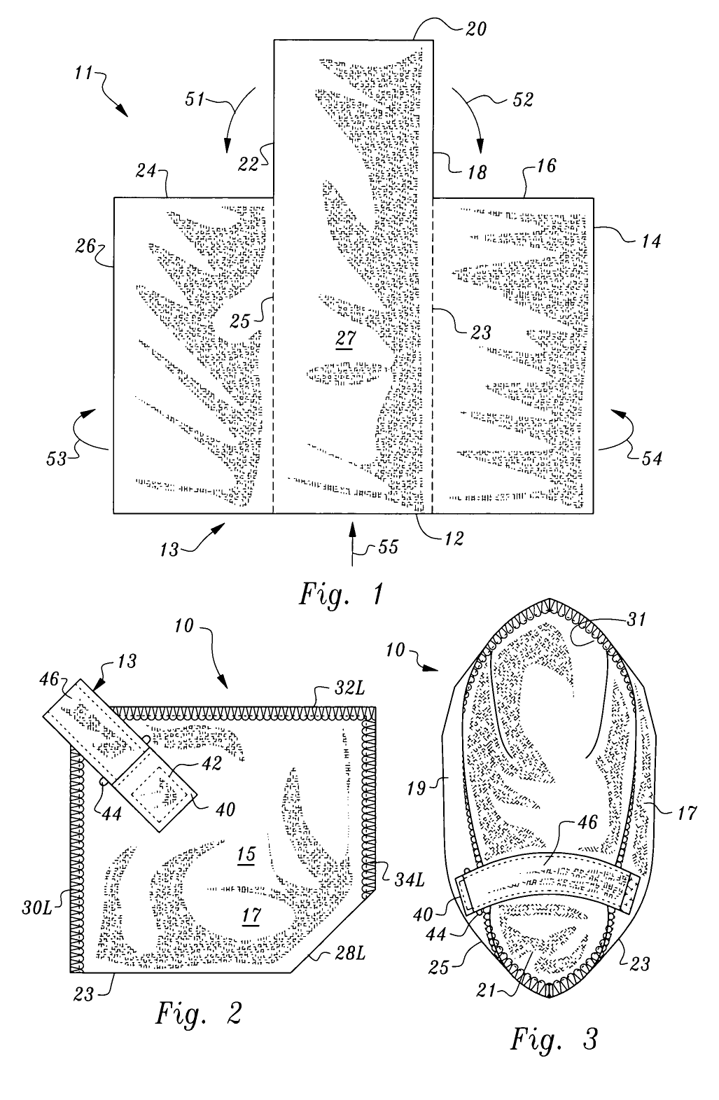

In FIG. 1 it is seen that the preferred mode of making the device of this invention is from an inverted T-shaped piece of cloth—the nature of which will be discussed infra. The generally accepted dimensions for a “one size fits all” device 10 is 9×9 with the removed corners being 3″×3″ thus yielding a Tee. The device is formed from a body portion 13 with an attached strap closure 13. Designator 12 refers to the front edge. The body portion 13 is formed by folding the front edge 12 upwardly into a U. The fabric being self-supporting, when the front edge 12 is folded, this operation brings edges 24 and 16 into a parallel arrangement with a base in between. Edges 26 and 14 are the left top and right top edges after this folding operation. While edges 24 and 16 represent the two rear edges subsequent to folding and reorientation, the edge 20 is the top rear surface, post assembly but pre-surging. Thus edges 22 and 18 are the rear vertical edges which become joined to edges 24 and 16 respectively during a sewing operation.

The folding operations and the optional surging or other binding operation, to prepare nice neat edges, will be explained during the course of the text presentation to a greater degree. However, the assembly arrows 51 and 52 indicate that these edges fold upwardly and as noted earlier, edge 12 folds upwardly into a U as shown by directional arrows 53 and 54 seen at the outer extremities of the FIG. 1. Thus edges 24-22 can mate and 18-16 can mate. Arrow 55 denotes an open area once edge 12 is folded as notated. Designator 27 is the bottom panel upon which the inserted foot heel rests. See also FIG. 10.

In FIG. 2 the assembled unit 10 is seen with its strap portion 13 and the body portion 15. Here the unit is deemed to be right closing, because the strap 46 passes through the D-loop 44 to close on the right side of device 10. The view here is of the left side of one of device 10. One device fits one heel, either the right or left heel of the wearer.

Left panel 17 is seen to have a surging binding on the front edge 30L, surging on the top edge 32L and surging 34 on the rear edge. The lower diagonal edge 28L is merely a fold line of the unitary structure's material.

Also seen in FIG. 2 is the strap portion 15 comprising strap 46, and the D-loop 44 through which it passes. D-loop 44 is retained by loop retainer 40, an elongated fabric strip over lay on itself, which is sewn into position along stitching 42, after passing through the D-loop 44.

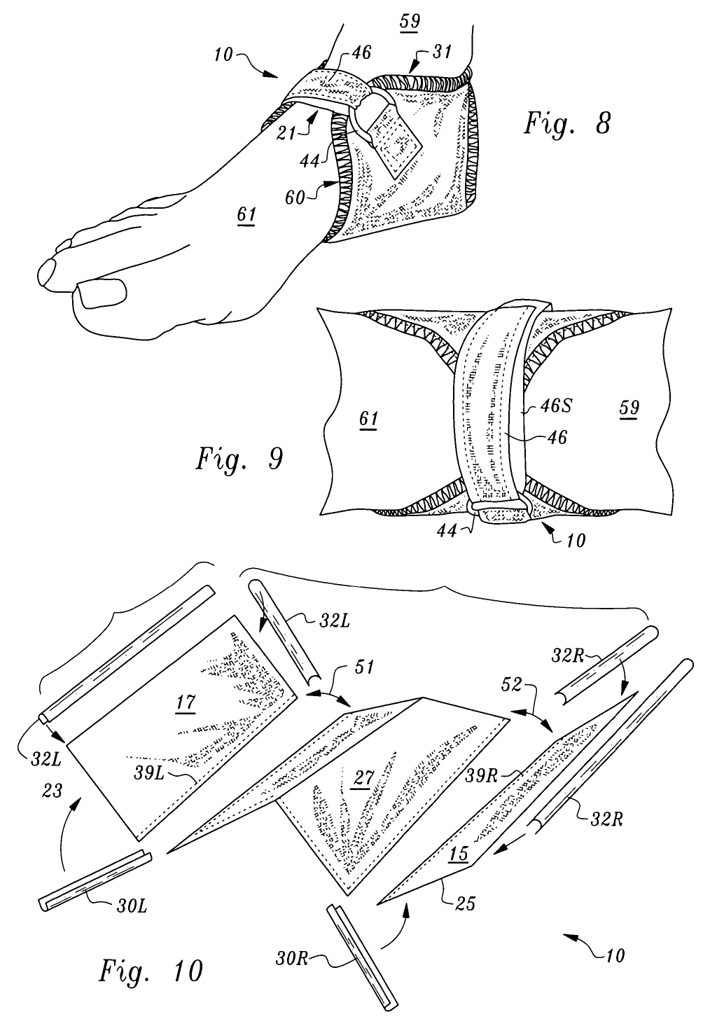

In FIG. 3 a top front perspective view of the device 10 is seen. Here the side panels 17 and 19 are shown in their respective position as are the two respective bottom folds 23 and 25. The wearer's foot heel area enters opening 21 while the ankle itself protrudes upwardly through top opening 31 and the toes of the wearer are not disposed within the device 10. See FIG. 8.

Since the unit shown in FIG. 3 is a left closing unit, the stitching 42 of loop retainer 40 is seen on the right side of the unit 10, but to the left of the viewer. D-loop 44 is also seen to be disposed on the right side of the unit, as is strap 46, which is shown in an engaged position such that the hook and loop fasteners sections are not seen in this view. Note that for ease of illustration no surging is depicted in this view.

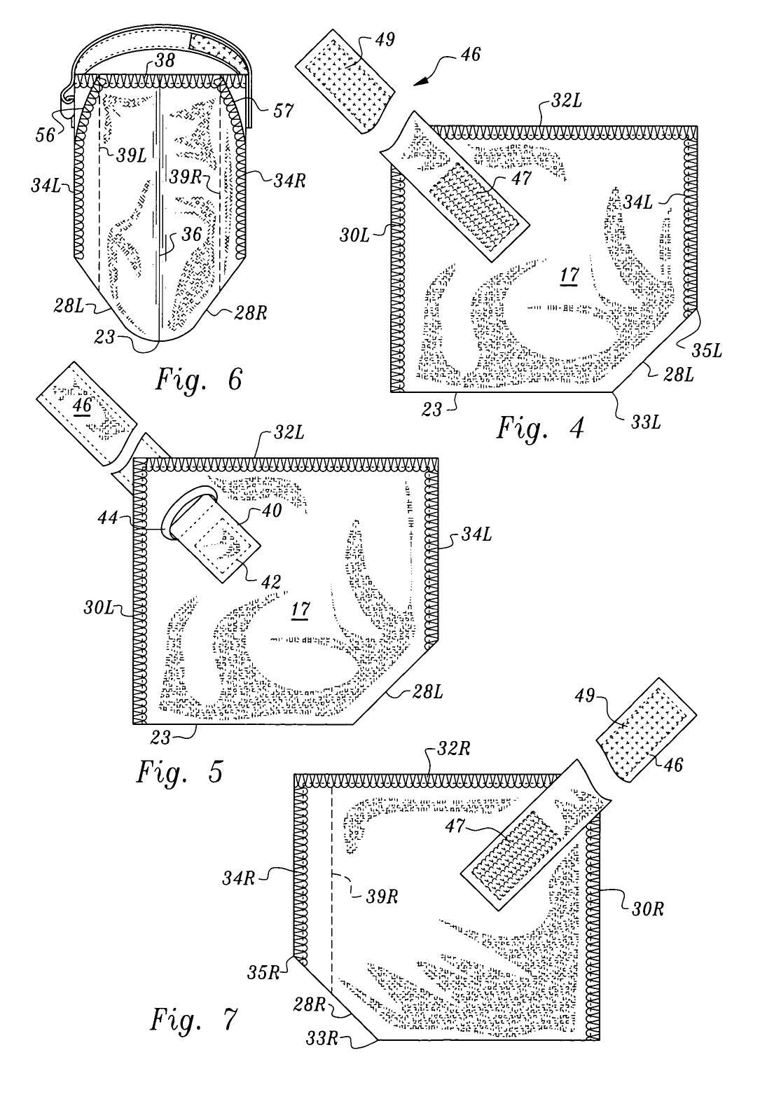

FIG. 4 is a left side elevational view of a left closing unit according to this invention. A heel protector is determined to be right or left closing based upon which side of the unit the strap 46's hook and loop fastener segments engage each other.

Here surging segment 32L of the left top panel, surging segment 34L of the rear edge of the left panel 17, and the front surge segment 30L are seen. More information about the surging procedure to bind the edges of the panels is recited infra with respect to FIG. 11. The right-hand panel is similarly surged, and the designators would be the same numerals but with an “R” thereafter instead of an “L.” Note that edge 28L is only an oblique fold line and that edge 23 is also only a fold line and therefore are not surged. Strap 46 is seen to be extended outwardly in order to reveal both its hook and loop fastener segments, wherein 47 is the hook pad and 49 the loop pad. The plastic or metal D-loop 44 through which the strap 46 passes is not seen in this view as it would be located on the right panel of this unit.

FIG. 5 is a left side elevational view of a right closing unit according to this invention. In accordance with the nomenclature set forth supra, it is seen that the closure segments disposed upon strap 46 are not seen in this view as they face away from the viewer. The loop retainer 40 is seen best in this view. It is a section of fabric an equal amount of which is folded over the D-loop 44 and under the loop 44 and then retained in position by stitching 42 that passes through both parts of the fabric section to the body panel 17. Such a sewing operation is deemed conventional.

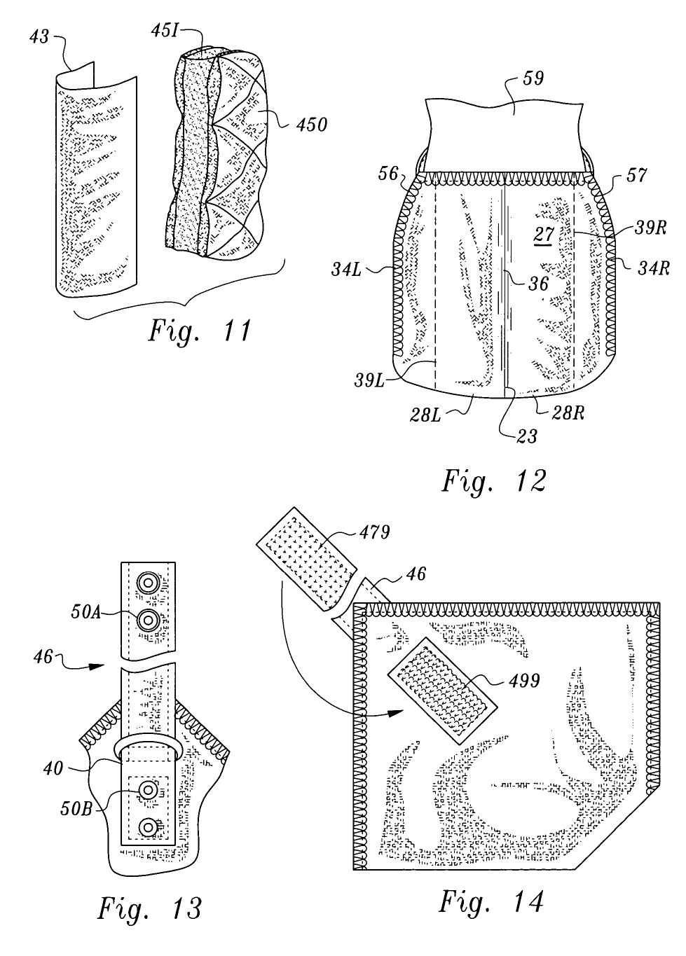

FIG. 6 is a rear elevational view of a device of this invention. The two oblique fold lines 28L and 28R are seen as is the inwardly disposed fold line 36. Surged edges 34L and 34R as well as surged top rear edge 38 are also seen. The dashed lines 39L and 39R are the stitching inset from the opposite edges of the rear panel 27, for attachment of the rear panel 27, to the respective side panels 17 and 19. See also designator 39R in FIG. 7. Such stitching is the primary attachment mode with the surging or other binding of the edges being the secondary attachment mode of the rear and side panels to each other. Note how the surging curves inwardly at about designator points 56,57 to permit the inwardly disposed fold 36 to open outwardly and top rear edge 38 to open flat upon insertion of a wearer's heel. See FIG. 12, which is a view similar to FIG. 6, but with a wearer's heel inserted into the device. This FIG. 12 will now be discussed out of order for ease of understanding. Note the distinction the two views, FIG. 6 is without the foot inserted and FIG. 12, the weight of the foot is in place causing the folds to unfold, ie. open up. In FIG. 12 the formerly oblique folded edges 28L and 28R, flatten out when the foot, not seen and the ankle of the wearer 59 are in position in the device 10. The bottom fold 23 becomes completely obliterated and lies flat along the rug or floor when the unit is disposed on a wearer. In essence a pleat formed by the folded oblique edges 28L and 28R and center fold 36 unfolds to yield the vertical upstanding back panel and part of the bottom panel. The weight of the foot causes the fold line 23 to expand and unfold which results in a visible shortening of the two side panels and the simultaneous formation of the base beneath the heel of the wearer. A comparison of the two figures at the locations noted is suggested.

FIG. 7 is a view of the right side of a right closing device according to this invention. This is the opposite side of the device shown in FIG. 5. Note that the corresponding surged edges to those surged edges previously discussed bear an “R” designation instead of an “L.” Here the strap 46 with its hook and loop pads—47 hook and 49 loop—are seen. These may be attached to the strap 46 fabric by either of two conventional attachment means, (1) stitching, or (2) by adhering a self-adhesive section of the fastener to the strap fabric. Note also the connection stitching line 39R which serves the purpose previously discussed.

In FIG. 8, the device 10 is seen to be disposed on a heel 60 of a wearer. Ankle 59 sticks out the top opening 31 while foot 61 sticks out of front opening 21. The overlay and engagement of strap 46 unto itself post passage through retainer D-loop 44 is readily seen.

Strap 46 can be made stronger by folding over a segment of fabric to double thickness and stitching it together prior to the attachment of the fastener elements thereto, as is seen by the presence of stitching line 46S in the view of FIG. 9, on a wearer's ankle. Passage of the strap 46 through the D-loop 44 is also seen in FIG. 9.

FIG. 10 depicts the assembly of device 10 at a frozen moment in time. Thus panels 15 and 17 are in the process of being folded upwardly into position to be stitched along what will be stitch lines 39L and 39R. All of the surge strips are seen located relative to the positions to where they are to be attached, as shown by the plurality of unnumbered arrows. The unit shown in this figure can be configured for either right or left closing as may be desired, since the strap and retainer loop have been omitted from the view intentionally for ease of understanding and illustration.

In FIG. 11, a closeup of the preferred fabric employed for this device and of the surging material is seen. Surging 43, a conventional product available in any fabric store, is sewn over the exposed edge of the non-woven polyester batting 45-I, disposed between two layers of washable quilting, 45-O. Such a material is readily available in the marketplace.

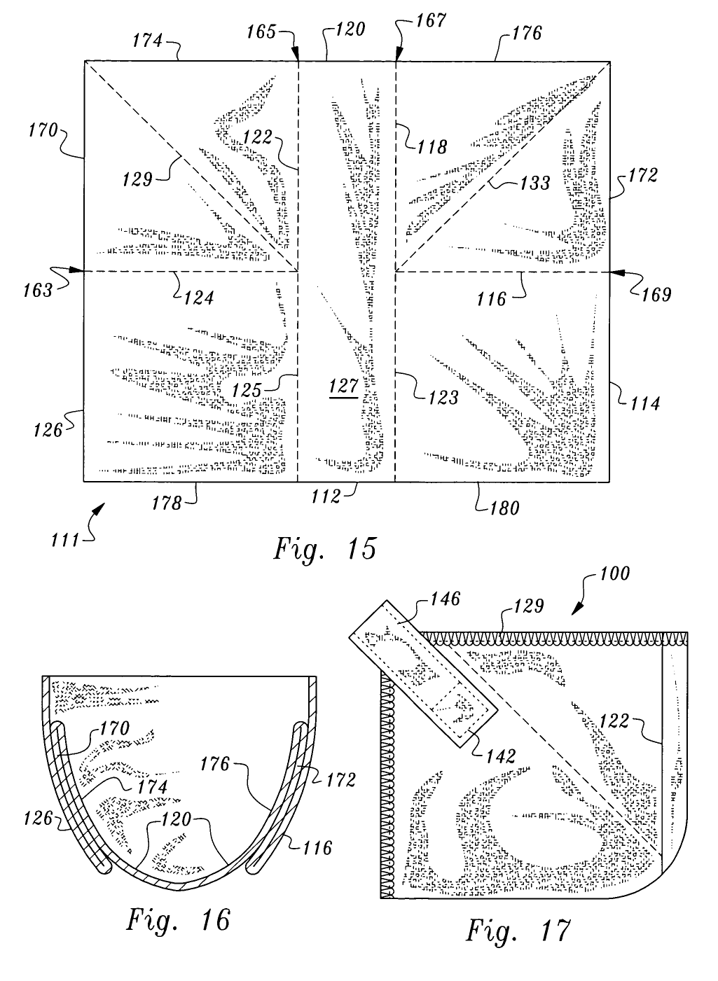

FIG. 14 is a side elevational view of a stitched only non-surged device that employs a simplified hook and loop closure. Here the retainer D-loop aforesaid is replaced by a sewn loop enclosure segment, 499 while the strap 46 includes only a sewn hook enclosure segment 479 for mating engagement with loop segment 499.

In all of the discussions above, it has been indicated that a back or rear panel 27 is present. Yet the views depicted in most of the figures show only left and right side panels joined together. See FIG. 3, and FIG. 7. In point of fact the rear panel is a pleat, which is internally folded such as not to be visible from the sides of the device. See FIG. 6. The rear fold 36 which separates the left part from the right part of the rear panel 27. This fold extends downwardly from the upper edge of the device, which if surged is edge 38, the full length of the elevation of the device, down to the point 33 on the bottom rear edge of each side panel. While 33R and 33L are depicted for ease of understanding of the respective side panels, they are in fact the same singular point 33. Two parallel oblique folds 28L and 28R extend from the termination point of the rear fold at point 33 (See FIG. 4) diverging upwardly but which are outward folds in contrast to fold 36, an inward fold, to points 35L (also FIG. 4) and 35R (see FIG. 7), taken in combination with fold 36 form an internal rear pleat which extends to flatten out and become the base and to become an extended rear panel, when the weight of the foot is placed on the pleat per FIG. 8. Refer again to FIG. 12 which shows the pleat in open position.

The discussion now moves to the preferred second embodiment of this invention. For ease and convenience of the reader, like numbers shall refer to like parts where applicable but the numbering shall be in the 100 series. Thus the reference numeral for the invention per se is 10 for the first embodiment. The invention is now referred to as 100 for the second embodiment.

It is also to be understood by the reader that the discussion concerning FIGS. 11 dealing with the surging technique and 13 dealing with alternative closure means are equally applicable to the second embodiment and need not therefore be repeated. Such is also true of the discussion showing a unit of this invention on a woman's foot.

As can be seen from FIG. 15, the second version of this invention is made from a full rectangle of fabric, about 8″ down and 9″ across, unlike the inverted Tee shaped member of FIG. 1 for the first version. In accordance with the nomenclature information supra, the front edge is 112, the right top edge of the device is denoted as 114, the left top is 126, while the left rear “A” is 122; the left rear “B” is 124 and the right rear “A” is 116 and the right rear “A” is 118. These “A & “B” designations are recited because upon folding an “A” edge and a “B” edge come together. Designators 123,125 and 127 are the bottom edge left, the bottom edge right and the rear panel respectively. Note the relationship to FIG. 1. The lines shown as dashed lines, both short and long are fold lines that arise during assembly. Designators 163,165, 167 and 169 are location points for the folding of the fabric member. Edges 170 and 172 are non-existent in the first embodiment and represent upper left and upper right fabric member vertical edges. Reference is now made to FIG. 16 in conjunction with FIG. 15.

To assemble the device of the second embodiment, one first finds point 163 along the left edge at about the midpoint of the fabric member. This is folded upwardly to point 165 which is spaced in about four inches, while fold line 129 is folded inwardly such that points 163 and 165 abut, such that top left edge 170 abuts top edge 174. Large dashed line 124 rotates from a horizontal location to become the folded rear left edge, adjacent wavy line 122. Left bottom edge 178 essentially rotates to become the left side edge of the assembled unit.

In FIG. 16, it is seen from the top that the panel edges prior to assembly but post folding are 126, 170 and 174 with 120 the middle of the top panel being in-between and forming the rear top edge post assembly.

The right side of the device is assembled in like fashion. Point 169 is moved to point 167, with line 133 folding inwardly and large dashed line 116 rotates from a horizontal location to become the folded rear left edge, adjacent wavy line 118. Right bottom edge 180 essentially rotates to become the right side edge of the assembled unit.

Since point 167 is also spaced in about four inches, the segment 120 between points 165,167 and wavy lines 122 and 118, becomes the rear wall of the device. The side rear edges 118 and 122 are not ironed or pressed into hard edges but are permitted to remain rounded. See also FIG. 16, on the right where the edges are seen to be 116, 172 and 176 in that order. Stitching is applied along wavy lines 126 and 118, and optionally along edges 174 and 176 to create the desired shape of the second embodiment. A surging step follows in the manner discussed previously along all exposed outer edges at the top and the vertical left and right edges, to yield the device shown in FIG. 17. See the discussion re FIG. 11, supra.

For ease of understanding hidden fold line 129 has been shown in this view. Note the location of wavy edge 122 upon assembly, but after the unit is pressed flat. The ACTUAL EDGE shown in FIG. 17 is the point shown in FIG. 16, which lies in the middle of rear surface 120. Note two lead lines in FIG. 16 to indicate the expanse of 120. Strap 146 is retained in position on the body of the device by stitching 142 also seen in this view. Suitable straps, [as well as strap 46] range from ¾ to one inch wide by about six inches in length from point of attachment, by stitching 42 or 142 for the respective embodiment.

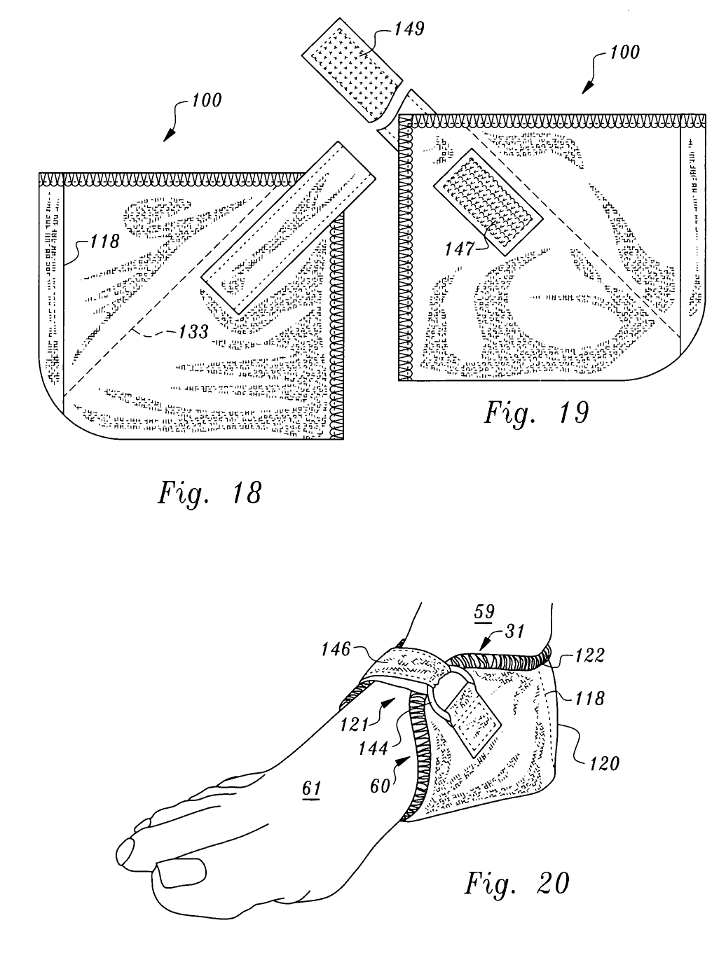

In FIG. 18 the opposite side of this second embodiment device is seen. Wavy line 118 is the counterpart to wavy line 122 for the opposite side. Rear 120 of the device falls between these two stitching lines. And, dashed line 133 is the counterpart of dashed line 129 previously discussed. Both of these are ironed flat for a more comfortable fit and feel of the device. Optionally stitching may be applied to retain these against the interior surface of the device, but such is not necessary, and to do so adds labor and the possibility of a rough thread for a tender foot.

Whereas FIGS. 17 and 18 depicted a right closing second embodiment, in FIG. 19 3a left closing unit is seen. But all other aspects of the second embodiment device remain the same.

Whereas in the first embodiment discussed, the strap went through a D-loop and closed back on itself, there is shown here a second simpler closure. Here the strap 146 has a loop pad 149 of a hoop and hook closure and the hook pad 147 is shown mounted on the opposite outside surface of the device. The use of a direct closing hook and loop closure without the separate feed through loop to achieve opposite side closing, may be utilized with both the second and the first embodiments interchangeably. Direct closing may take place on either the right or left side surface of the device. Note further that indirect closing utilizing the loop structure discussed with respect to the first embodiment, may be employed for the second embodiment as well; again with the closing being on either side panel of the device.

Thus reference is made to FIG. 20, a view similar to FIG. 8. Here a second embodiment device is seen on the foot of a female, but the second embodiment has an indirect closure with the strap passing through loop 144. The second embodiment is worn the same way with the foot going into opening 121 and the strap extending across the instep. Also note how the stitching line along 122 defines the left rear edge of the device, and stitching line 118 defining the right rear edge.

The dimensions of 8″ by 9″ may be varied slightly for females small in foot size, say size 5 and under, and increased slightly for women whose feet are greater than average.

While the device is directed primarily to females, it can also be used by men who may suffer from cracked heels and related skin problems as well.

As mentioned earlier the devices of this invention may be either left closing or right closing, the actual designation being the side of the device upon which the closure engagement transpires. In the manufacture of the devices, one might choose to have a right closing unit for the right foot and a left closing foot for the left foot; or both feet may utilize units that close on the respective same side of the foot either right or left.

The devices of this invention may be made in any color, though dark is preferred over light colored fabric such that soiling is not readily visible from repeated wearing.

The big advantage of the preferred fabric which is a double layer of quilting with a polyester batt interposed is that devices made from the preferred material are substantially not susceptible to bleed-through of medicaments. That is, balms and petroleum jelly are substantially not absorbed by the fabric as so they do not pass through the non-woven batting interposed between the quilted layers. Thus the medicament can be substantially totally absorbed by the cracked heel of the wearer to provide relief from the condition suffered by the wearer. These devices can be worn individually or in pairs as may be required. They also permit the wearer to be medically treated while sleeping without the fear of staining expensive linens. Of course, they may be worn without the heel being medicated, merely to prevent irritation to dry, chapped or irritated heels on both the vertical and horizontal surfaces thereof. Of course other materials that do not permit medicaments to pass through to socks, bedding and shoe linings, may be utilized for the devices of this invention. Such fabrics are available in the marketplace.

It is seen that I have provided a new device that can be worn with or without medication on the cracked heels of the wearer. The device is easy to slip into and is secured by passing a strap through a retainer loop and overlaying the strap back over itself. While Velcro brand or an equivalent hook and loop fastener is the preferred closure, one or more of mating two-part snaps such as 50A that mattingly engages 50B as shown in FIG. 13 may also be employed

While surging the edges of the fabric has been discussed, it is not a required step. Other bindings, as well as no binding may be used along the edges of the batt lined quilting material employed herein to inhibit the spread of medicament and provide comfort to the wearer. It has been fond that the maximum comfort during wear is achieved if the strap is attached at about a 45-degree angle, rather than truly horizontally across the instep.

Since certain changes may be made in the described apparatus without departing from the scope of the invention herein involved, it is intended that all matter contained in the above description and shown in the accompanying drawings shall be interpreted as illustrative and not in a limiting sense.

I claim:

1. A fabric device to be worn over the heel of a user to protect a cracked or inflamed heel, whether being medically treated or not, to protect the heel from further injury and discomfort, which device comprises a main body portion having an open top, an open front, a left side and an equally sized right side, a folded base joined to each of the two sides, and to a rear, which base is defined and which rear, both unfold when a foot is placed in the open top and open front;
and a strap portion having a closure thereon to secure the device to the heel of the wearer.

2. A fabric device to be worn over the heel of a user to protect a cracked or inflamed heel, whether being medically treated or not, to protect the heel from further injury and discomfort, which device comprises a main body portion of a Tee-shaped fabric section which section is folded and stitched to form a left side panel and a right side panel, with a back panel comprising an internal pleat which pleat is both vertically and obliquely connected to both of said right and left side panels,
said device having an open front and an open top;
whereby when the weight of a wearer's heel is placed into the device, the internal rear pleat spreads apart and is opened to create a rear panel and a base between the side panels.

3. The device of claim 2 further including a strap attached to one side panel.

4. The device of claim 17 further including a strap having a closure thereon is attached to one side panel.

5. The device of claim 3 wherein the strap includes a closure thereon.

6. The device of claim 2 wherein a D-loop is retained in place on one side panel by a sewn in place loop retainer, through which a strap can pass.

7. The device of claim 3 further including a hook and loop closure disposed upon said strap.

8. The device of claim 7 wherein the loop and retainer are mounted at about a 45-degree angle on the left panel of the device.

9. The device of claim 6 further including a two-part snap closure disposed on the strap.

10. The device of claim 2 wherein the front, top and rear edges of each of the side panels is overlaid with a binding.

11. The device of claim 10 where in the binding is surging.

12. The device of claim 2 wherein the material used for the device is quilted with a polyester batt between the two layers of fabric.

13. A fabric device to be worn over the heel of a user to protect a cracked or inflamed heel, whether being medically treated or not, to protect the heel from further injury and discomfort, which device comprises a main body portion of a Tee-shaped fabric section which section is folded and stitched to form a left side panel and a right side panel, with a rear panel comprising an internal pleat which pleat is obliquely connected to both of said right and left side panels at the respective lower ends thereof,
said device having an open front and an open top;
whereby when the weight of a wearer's heel is placed into the device, the internal rear pleat spreads apart and is opened to create a rear panel and a base between the side panels;
and wherein a loop is retained in place on one of said left and right side panels by a sewn in place loop retainer, through which a strap can pass, and a strap having a closure thereon is mounted on the other of said left and right side panels.

14. The device of claim 13 wherein the strap and a D-loop retainer are each mounted at about a 45-degree angle.

15. The device of claim 14 wherein the material used for the device is quilted with a polyester batt between the two layers of fabric.

16. A fabric device to be worn over the heel of a user to protect a cracked or inflamed heel, whether being medically treated or not, to protect the heel from further injury and discomfort, which device comprises a main body portion having an open top, an open front, a left side and an equally sized right side, a base joined to each of the two sides, and an inwardly folded pleated rear, which rear unfolds to expand and define a rear when a foot is placed in the open top and open front;
and a strap portion having a closure thereon to secure the device to the heel of the wearer.

17. A fabric device to be worn over the heel of a user to protect a cracked or inflamed heel, whether being medically treated or not, to protect the heel from further injury and discomfort, which device comprises a main body portion having an open top, an open front, a left side and an equally sized right side, a folded base joined to each of the two sides, and to an outwardly folded rear, which base and rear unfold to expand and become defined when a foot is placed in the open top and open front;
and a strap portion having a closure thereon to secure the device to the heel of the wearer.

18. The device of claim 17 wherein the front, and the top edges of each of the side panels is overlaid with a binding.

19. The device of claim 18 where in the binding is surging.

20. The device of claim 17 wherein the strap and a D-loop retainer are each mounted at about a 45-degree angle; and further
wherein the material used for the device is quilted with a polyester batt between the two layers of fabric.
